(19) United States
(12) Patent Application Publication
(10) Pub. No.: US 2006/0270989 A1
(43) Pub. Date: Nov. 30, 2006

(54) Gastric fastening system

(76) Inventors: Donald J. McMichael, South Jordan, UT (US); Edward B. Madsen, Riverton, UT (US); Nathan V. Shirley, Herriman, UT (US)

Correspondence Address: KIMBERLY-CLARK WORLDWIDE, INC., 401 NORTH LAKE STREET, NEENAH, WI, 54956, US

(21) Appl. No.: 11/139,927

(22) Filed: May 27, 2005

(51) Int. Cl. A61M 5/178

(52) U.S. Cl. 604/164.04; 604/910

(57) ABSTRACT

A gastric fastening system is disclosed. The system contains a cannula for placement into a body lumen within a body. The cannula has a first end for positioning within the lumen, a collapsible region proximal to the first end for placement within the lumen, and an actuator for causing the collapsible region to collapse outwardly from the cannula so as to form a radial collar having a second diameter greater than the first diameter while foreshortening the length of the cannula. The collar secures the cannula against extraction from the lumen.

### BACKGROUND OF THE INVENTION

The present invention relates to an internal anchoring device and, more particularly, to a internal anchoring device for percutaneously placing various catheters such as: gastrostomy and/or Jejunostomy tubes.

For example, numerous medical conditions exist in which it becomes necessary to gain percutaneous access to viscera such as the stomach or small intestines. Situations where a patient has lost the ability to swallow and will require long term nutritional support may dictate feeding directly into the stomach or jejunum. This type of feeding may be accomplished by inserting a feeding tube into the patient's stomach such that one end remains anchored in the stomach, while the other end remains external to the patient's body for connection to a nutrient source.

Feeding tubes may be inserted into a patient's stomach in a number of ways. Feeding tubes may be endoscopically placed, surgically placed through an open incision, laproscopically placed, or percutaneously placed under endoscopic, fluoroscopic or ultrasonic guidance.

Different types of feeding tubes may be placed using these procedures, examples include Gastrostomy, Jejunostomy or Gastro-Jejunostomy. These tubes may be retained in the lumen (stomach or intestine) with a variety of retention anchors. These anchoring mechanisms include: inflatable balloons, obturatable domes, fixed dome-type bumpers, or suture wings.

When placing a tube with an inflatable balloon percutaneously, it is preferred to perform a gastropexy procedure during placement. This procedure enables the physician to attach the visceral wall to the abdomen. This attachment is critical to prevent inadvertent separation and exposure of the peritoneal cavity to contamination and possible peritonitis.

The anchoring mechanism of the prior art devices typically consist of a small metal t-shaped fastener that can embed into the gastric or intestinal wall and ultimately lead to infection. The t-shaped fastener or t-bar is not removable and is left in the body cavity where it is allowed to pass naturally in the patient's stool. In many cases the t-bar is not passed and remains within the body cavity. Moreover, the t-bar has sharp edges which can be uncomfortable for the patient.

What is needed is a fixation device that is easy to place within an internal body cavity, allows for the formation of a stoma between the internal body cavity and the external environment, and enables the user to easily remove the fixation device when it is no longer necessary.

### SUMMARY OF THE INVENTION

In response to the foregoing problems and difficulties encountered by those of skill in the art, the present invention is directed toward a gastric fastening system. In one aspect of the invention, the gastric fastening system may include a placement cannula having a first and a second end. The placement cannula would be adapted to percutaneously enter a body lumen. A secondary cannula may also be provided. The secondary cannula would be removably associated with the placement cannula and may be internally engaged with the placement cannula in a sliding relationship. The secondary cannula would have an actuatable collar portion capable of axial collapse and radial expansion when actuated. The collar portion would be adapted for placement within the body lumen so as to secure the secondary cannula within the lumen. An actuator for manipulating the collar portion would be provided. The actuator would be used for actuating or manipulating the collar portion from a first deactuated position to a second actuated position and vice versa. The system may include a trocar at one end of the placement cannula for percutaneously entering the body lumen thus eliminating the need for a previously placed surgical incision.

The collar portion disposed upon the secondary cannula may be formed by slitting the secondary cannula in a plurality of locations. In one embodiment the slits may be radially disposed about the circumference of the cannula and extend along a portion of the length of the cannula. Such a configuration would create a plurality of wings axially extending along the length of the cannula from a first end point to a second end point. The wings would be radially disposed about the circumference. Each wing would have a first and a second attachment with respect to the secondary cannula. These attachments would correspond to the first and second end points respectively whereupon actuation of the collar portion by the actuator moves the first and second attachments toward one another causing each wing to hinge outwardly between the first and second attachments.

In another aspect, the actuatable collar portion may have a slitted section of the secondary cannula located proximal to a first end of the cannula. Each slit would extend axially along the cannula from a first end to a second end. The first and second ends would be movable with respect to one another between a first position and a second position. The collar portion in the second position would be axially collapsed and radially expanded with respect to the first position.

In still another aspect, the actuatable collar portion may contain a plurality of hinged elements, each having a first and a second attachment to the secondary cannula, and each movable between a first extended position and a second collapsed position by actuation of the actuator. In yet other aspects of the invention, the actuatable collar portion may be elastically deformable, may contain a plurality of necked regions circumferentially disposed about the cannula, and may contain a plurality of perforations disposed about the cannula which would form the collapsible region. An elastically deformable collar portion may be created by encasing the hinged elements in an elastomeric sheath or sleeve. For example, a silicone sleeve may be placed over the wings resulting in the creation of a silicone flange once the collar portion is actuated. Such a device might aid in patient comfort as it would reduce the possibility of the flange or collar embedding into the gastric mucosa. For similar reasons, it might also foster eventual collapse of the wings during the removal process.

The actuator may contain a wire having a catch at one end. The catch would be adapted to capture a portion of the secondary cannula and axially displace a distal end of the secondary cannula toward a proximal end of the secondary cannula. This would cause the axial collapse and radial expansion of the collar portion.

In yet another aspect, the invention may incorporate a locking hub that works in conjunction with the collar portion to secure the secondary cannula within the lumen.

Other objects, advantages and applications of the present invention will be made clear by the following detailed description of a preferred embodiment of the invention and the accompanying drawings wherein reference numerals refer to like or equivalent structures.

### BRIEF DESCRIPTION OF THE DRAWINGS

FIG. 1 is a perspective view of one embodiment of the present invention being placed into a patient's internal body lumen;

FIG. 2 is a perspective view of the FIG. 1 embodiment depicting use of an actuator in accordance with the present invention;

FIG. 3 is a perspective view of the FIG. 1 embodiment in a collapsed position in accordance with the present invention;

FIG. 4 is a perspective view of the collar portion of the FIG. 1 embodiment;

FIG. 5 is a perspective view of a locking hub in use with the FIG. 1 embodiment; and

FIG. 6 is a perspective view of the FIG. 1 embodiment depicting the addition of an elastomeric sheath over the FIG. 4 collar portion.

### DETAILED DESCRIPTION OF THE PRESENT INVENTION

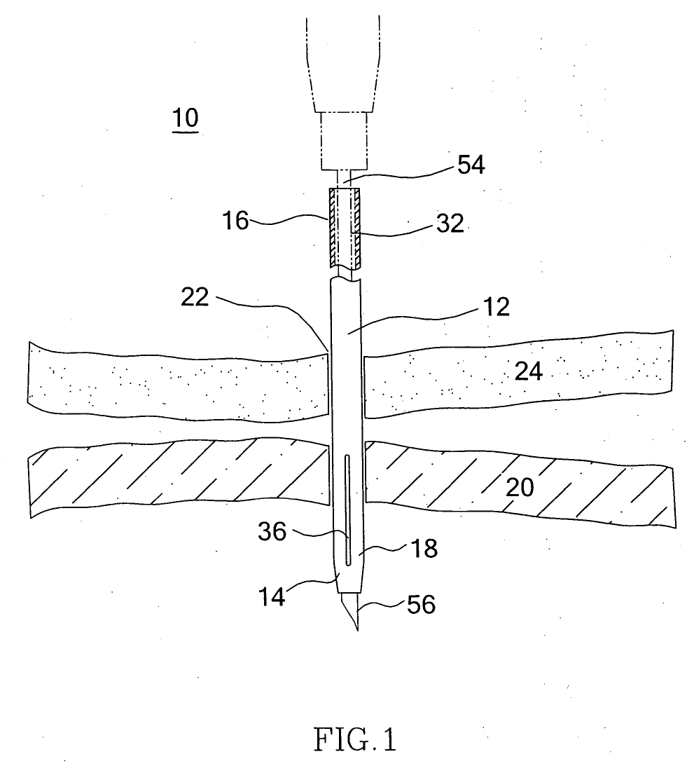

In response to the foregoing challenges that have been experienced by those of skill in the art, the present invention is directed toward a gastric fastening system 10 for use in facilitating initial placement of enteral feeding tubes and the like as shown in the FIGs. In one embodiment, as depicted in FIG. 1, the system 10 includes a cannula 12 having a first end 14 and a second end 16. Disposed between the ends, proximal to the first end 14, is a collapsible collar portion 18. The cannula 12 is designed to be percutaneously placed into a body lumen 20, for example a stomach. More specifically, the first end 14 of the cannula 12 is inserted through an incision 22 in a patient's abdomen 24 and introduced into the stomach lumen 20. As depicted, a placement cannula 54 is slidingly engaged with the cannula 12. The placement cannula 54 may be inserted into an internal lumen 32 running through the cannula 12 and may contain a trocar 56 at its tip. The trocar 56 may be used to percutaneously pierce the skin and tissue of the body lumen 20 and abdomen 24.

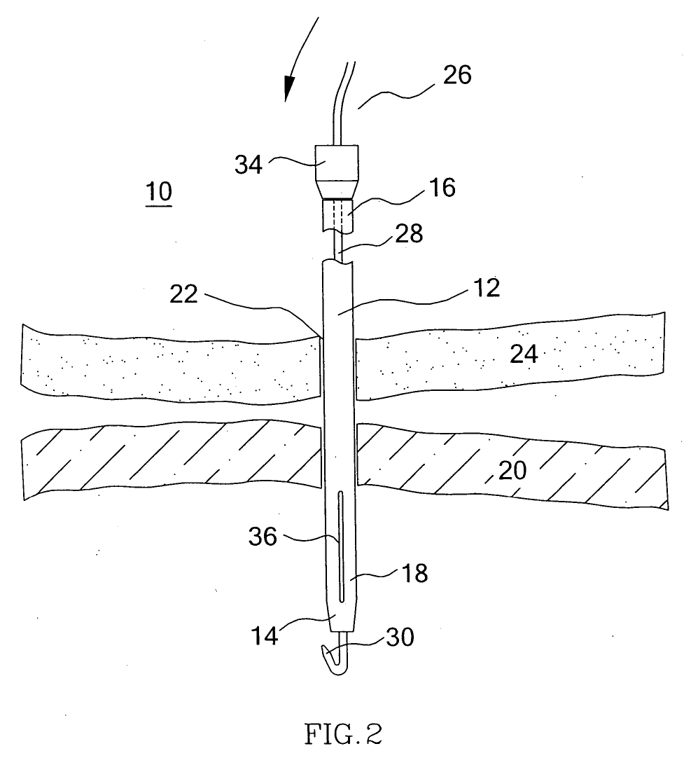

After the collar portion 18 enters the lumen 20 and the cannula 12 is otherwise located in the desired position, the placement cannula 54 is designed to be withdrawn from the cannula 12 leaving the cannula 12 in place. At that time, an actuator 26 may be introduced into the cannula 12 or otherwise engaged with the cannula 12 as shown in FIG. 2. In one embodiment, the actuator 26 may be a wire or cable 28 having a hook or catch 30 designed to engage a portion of the cannula 12. For example, the cable 28 may be led or fed through the internal lumen 32 within the cannula 12 until the catch 30 extends beyond the first end 14 of the cannula 12. A stop 34 may be present on the cable 28 to indicate when the catch 30 is in the desired position. Of course this configuration would be easily accommodated by placing the stop 34 a slightly greater distance from the catch 30 on the cable 28 than is the distance between the first and second ends 14 and 16 respectively of the cannula 12.

In any event, once the catch 30 protrudes beyond the first end 14, the feed direction of the cable 28 is then reversed causing the catch 30 to engage the first end 14. Continued pulling on the cable 28 causes the collapse or activation of the collar portion 18. In many embodiments, the first end 14 may be tapered as shown in FIGS. 1 and 2 so as not to cause the catch 30 to interfere with the patient's tissue upon removal of the apparatus. Though the catch 30 is depicted as a single hook in FIG. 2, this configuration is not required. A dual prong or triple prong hook or some other mechanism known to those having skill in the art would work as well and should be readily ascertainable to one of skill in the art without resort to a drawing. The hook portions may be spring biased so that once they protrude from the first end 14, they spring outward so as to catch on the first end. The tapered first end, in this embodiment, would serve to minimize tissue injury upon eventual withdrawal of the cannula from the lumen 20.

Moreover the catch is not required to protrude from the cannula 12. An alternative arrangement may comprise having a mechanism internal to the lumen 32 adapted to engage with the catch 30. Though such a mechanism is not depicted, such things as a tabs, grooves, loops, a mating hook, and slots comprise just a few of the possibilities. These and variations on these are merely intended as a list of some of the possible variations available to those of skill in the art and are not meant as limitations. These too should be readily ascertainable to one of skill in the art without resort to a drawing.

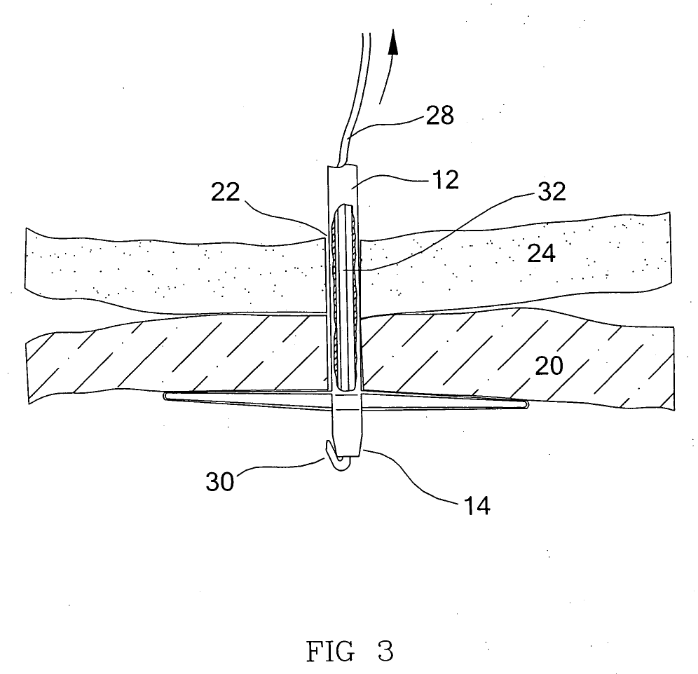

In any event, turning now to FIG. 3, it may be seen that by manipulation of the actuator 26, the collar portion 18 can be made to collapse such that the first end 14 of the cannula 12 is effectively drawn toward the second end 16. The collar portion 18, in one embodiment as shown on FIG. 4, may simply comprise a slitted section of the cannula 12. More specifically, a plurality of such slits 36 may be radially disposed about the circumference 38 of the cannula 12. The slits 36 may be made to extend axially along the length of the cannula 12 from a first end point 40 to a second end point 42. Each slit 36 may generally be of the same length and begin and end generally at the same end points radially about the circumference 38. The slits 36, in this embodiment form sections or wings 44. The wings 44 comprise those individual segments of the cannula 12 that lie between the slits 36 and in the FIG. 4 embodiment may be seen to be equally spaced about the central axis 46 of the cannula 12 around the circumference 38. Circular or other shaped apertures 48 may be included at the end points 40 and 42 to serve as stress relievers but are not necessary to practice the invention. These apertures 48 are shown at one end of the FIG. 4 illustration by way of illustration. Typically such apertures would be placed at each end point 40 and 42, however, there is no requirement that they be present or that they be present at each end point.

Looking back to FIG. 3 in conjunction with FIG. 4, it may be envisioned that the first end point 40 is being drawn toward the second end point 42. In this configuration, the slits 36 and wings 44 are adapted to accommodate this movement. The slits 36 allow the wings 44 to bend or otherwise deform. Such deformation can be made to occur along the entire length of the wing 44 or may be focused at one or more weakened, thinned, or necked locations 50. Each wing 44 will hinge outwardly at the necked location 50 such as that shown substantially mid-length along the wing 44, thus effectively collapsing the collar portion 18 reducing the effective length of the cannula 12 while axially expanding the collar portion 18 to a second diameter larger than the diameter of the cannula 12. This configuration should be easily understood by those skilled in the art and for all practical purposes, prevents the cannula 12 from being pulled from the body lumen 20.

To lock the cannula 12 in place, while retaining the collar portion 18 in its axially collapsed state requires some means of keeping the actuator 26 engaged or otherwise preventing the collar portion 18 from returning to its disengaged or deactuated condition. Some means that may be found suitable for this purpose consist of a locking device or devices that engage a portion of the cannula 12 protruding from the body. The locking device must also secure the actuator 26 as well, typically securing the actuator 26 in place on the cannula 12. In one embodiment, the stop 34 may be made to be adjustable along the length of the cable 28 so that once the collar portion 18 is in the desired position, the stop 34 may be slid along the cable 28 until it contacts the second end 16 of the cannula 12 thus preventing any of the cable 28 that may be protruding from the cannula 12 from entering the lumen 20.

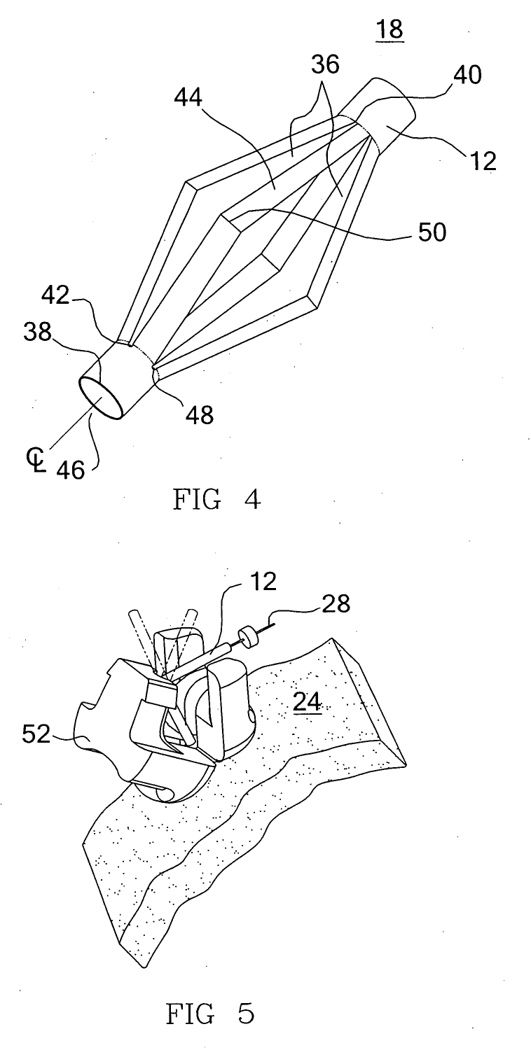

In another embodiment, as depicted in FIG. 5, the locking device may consist of a locking hub 52 that clamps the cannula 12 and cable 28 preventing one from moving with respect to the other. A locking hub such as that disclosed in co-pending application filed on May 27, 2005 under US Express Mail Number EV064854695US to McMichael et al., which is incorporated herein by reference in its entirety. In this embodiment, the cannula 12 may be made to be bendable or otherwise deformable so that the locking hub 52 is capable of deforming and/or crimping the cannula 12 against the cable 28. Such a cannula may be made of numerous deformable biocompatible materials including, but not limited to, materials such as polyolefins, pvc, polyurethanes and silicone. Other embodiments may of course be utilized and would be understood by those skilled in the art. An important aspect of embodiments similar to that depicted in FIG. 5 would be to ensure that the cable 28 or other actuator mechanism 26 is captured and prevented from inadvertent movement.

In use, one or more of the systems 10 are employed. In many of the embodiments, the cannula 12 has an external diameter not exceeding 0.075 inch (approx. 1.9 mm) therefore use of multiple systems may be warranted without creating undue burden on or to the patient. In use with respect to gastrointestinal procedures, typically the placement cannula 54 is engaged with the cannula 12. Both cannulae 12 and 54 are percutaneously introduced through the abdomen wall 24 and into the stomach lumen 20. The incision 22 is made by the trocar 56 located at the end of the placement cannula 54. The stomach lumen 20 would likely be insufflated so as to ease placement. A luer fitting (not shown) may be disposed on the end 16 so that contrast may be introduced and detected by appropriate means so as to ensure that the first end 14 of the cannula 12 is located as desired. Once the cannula 12 is placed, the placement cannula 54 and trocar 56 are removed. The actuator 26 is introduced or otherwise activated as described above and the collar portion 18 of the cannula 12 is axially collapsed thereby preventing inadvertent removal from the stomach lumen 20. The locking mechanism such as the locking hub 52 is engaged with the cannula 12 and may be place adjacent the patient's abdomen thereby securing the cannula 12 and actuator 26 in place minimizing axial movement of the cannula 12 either into or out of the stomach lumen 20.

Such a procedure may be desirable during a gastropexy procedure, where it is desirable to adhere the stomach to the inside of the abdominal wall to enable the creation of an artificial stoma. By pulling the wings 44 comprising the collar portion 18 a desired amount the stomach and abdominal walls are brought into contact and sutured to one another until such time that the stoma has healed, thereby sealing both the stomach or the external environment from the peritoneal cavity. Removal of the locking mechanism allows the wings 44 to relax, thereby permitting the cannula 12 to be withdrawn from the patient. This eliminates the need for removing the retaining mechanism (wings 44 in the FIGs.) endoscopically or cutting the retaining mechanism free of the cannula and allowing it to pass through the gastrointestinal tract.

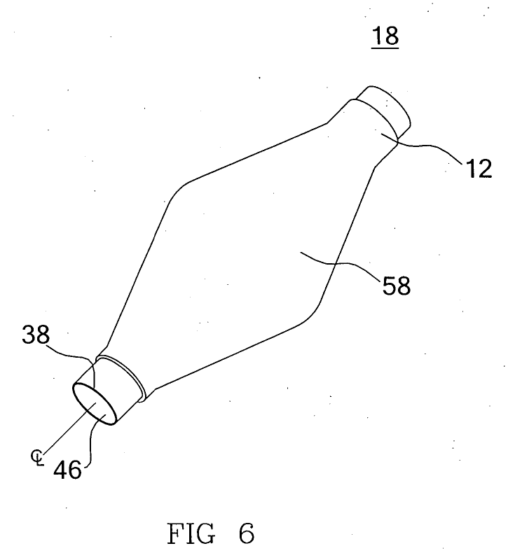

Other embodiments that may prove useful include enabling the collar portion 18 to be elastically deformable through its construction or through the application of an elastomeric material that may be conjoined to the collar portion. In such embodiments, the collar portion may comprise a plurality of perforations disposed about the cannula, a plurality of necked portions that are collapsible, or a number of other embodiments. For example, as shown in FIG. 6, the actuatable collar portion may be similar in construction as the FIG. 4 embodiment with the addition of an elastomeric sleeve or sheath 58 placed over the wings 44. This sleeve 58 may be a silicone or other elastomeric material and if placed over the wings 44 would result in the creation of an elastomeric flange once the collar portion 18 was actuated. Such a device might aid in patient comfort as it would reduce the possibility of the flange or collar embedding into the gastric mucosa. For similar reasons, it might also foster eventual collapse of the wings during the removal process. Such embodiments would be understood by those skilled in the art. An important aspect to these embodiments is that the cannula be capable of reversible and selective axial shortening by foreshortening of the collar portion while radially expanding it as well

As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

We claim:

1. A gastric fastening system comprising:
a placement cannula having a first and a second end, the placement cannula adapted to percutaneously enter a body lumen;
a secondary cannula with which the placement cannula is removably associated, the secondary cannula having an actuatable collar portion capable of axial collapse and radial expansion when actuated, the collar portion adapted for placement within the lumen to secure the secondary cannula within the lumen; and
an actuator for manipulating the collar portion from a first deactuated position to a second actuated position.

2. The system of claim 1 wherein the placement cannula is slidingly engaged within a lumen through the secondary cannula.

3. The system of claim 1 comprising a trocar at a first end of the placement cannula for percutaneously entering the body lumen.

4. The system of claim 1 wherein the secondary cannula comprises a circumference, a length beginning at a first end and terminating at a second end, and a central axis along the length, the collar portion being located proximal to the first end and comprising a plurality of spaced apart slits, the slits being radially disposed about the circumference.

5. The system of claim 1 wherein the secondary cannula has a length and a circumference, and the collar portion comprises a plurality of wings axially extending along the length from a first end point to a second end point, the wings radially disposed about the circumference.

6. The system of claim 5 wherein each wing comprises a first and a second attachment with the secondary cannula at the first and second end points respectively, whereupon actuation of the collar portion by the actuator moves the first and second attachments toward one another causing each wing to hinge outwardly between the first and second attachments.

7. The system of claim 1 wherein the actuatable collar portion comprises a slitted section of the secondary cannula located proximal to a first end of the cannula, each slit extending axially along the cannula from a first end to a second end, wherein the first and second ends are movable with respect to one another between a first position and a second position, and the collar portion in the second position is axially collapsed and radially expanded with respect to the first position.

8. The system of claim 1 wherein the actuatable collar portion comprises a plurality of hinged elements, each having a first and a second attachment to the secondary cannula, and movable between a first extended position and a second collapsed position by actuation of the actuator.

9. The system of claim 1 wherein the actuator comprises a wire having a catch at one end, the catch adapted to capture a portion of the secondary cannula and axially displace a distal end of the secondary cannula toward a proximal end of the secondary cannula thereby axially collapsing and radially expanding the collar portion.

10. The system of claim 1 wherein the actuatable collar portion is elastically deformable.

11. The system of claim 1 comprising a locking hub working in conjunction with the collar portion to secure the secondary cannula within the lumen.

12. A gastric fastening system comprising:
a cannula for placement into a body lumen within a body, the cannula having a first diameter, a first end for positioning within the lumen, a collapsible region proximal to the first end for placement within the lumen, a second end for positioning external to the body, and a length defined by the distance between the first and second ends; and
an actuator for causing the collapsible region to collapse outwardly from the cannula so as to form a radial collar having a second diameter greater than the first diameter while foreshortening the length of the cannula, the collar securing the cannula against extraction from the lumen.

13. The system of claim 12 comprising a plurality of radially disposed slits axially oriented along the length of the cannula so as to form the collapsible region.

14. The system of claim 12 comprising a plurality of necked regions circumferentially disposed about the cannula so as to form the collapsible region.

15. The system of claim 12 comprising a plurality of perforations disposed about the cannula so as to form the collapsible region.

16. The system of claim 12 wherein the actuator is inserted into the cannula from the second end whereupon manipulation of the actuator collapses the collapsible region outwardly.

17. The system of claim 12 wherein the actuator comprises a wire passed through the cannula from the second end and having a catch adapted to engage the first end of the cannula so that pulling upon the wire draws the first end toward the second end thereby collapsing the collapsible region.

18. The system of claim 12 comprising a locking hub for placement upon the cannula proximal to the second end so as to secure the cannula in the lumen.

19. The system of claim 12 wherein the collapsible region comprises a plurality of hinged elements, each having a first and a second attachment to the cannula, and movable between a first extended position and a second collapsed position by actuation of the actuator.

20. The system of claim 12 wherein the collapsible region comprises an elastically deformable material.
